(19) United States
(12) Patent Application Publication
(10) Pub. No.: US 2024/0360936 A1
(43) Pub. Date: Oct. 31, 2024

(54) STERILE CONNECTION MEMBER AND STERILE CONNECTION APPARATUS

(71) Applicant: WUXI NEST BIOTECHNOLOGY CO., LTD, WUXI (CN)

(72) Inventors: Feng CHEN, WUXI (CN); Weidong YANG, WUXI (CN)

(73) Assignee: WUXI NEST BIOTECHNOLOGY CO., LTD, WUXI (CN)

(21) Appl. No.: 18/770,672

(22) Filed: Jul. 12, 2024

Related U.S. Application Data
(63) Continuation of application No. PCT/CN2022/139654, filed on Dec. 16, 2022.

(30) Foreign Application Priority Data
Aug. 15, 2022 (CN) 202210975773.6

(51) Int. Cl. F16L 37/098

(52) U.S. Cl. CPC F16L 37/098; F16L 2201/44

(57) ABSTRACT

Disclosed are a sterile connection member and a sterile connection apparatus. The sterile connection member includes a main body. A first surface of the main body is connected to an external pipeline. A fluid channel is arranged inside the main body. The fluid channel penetrates from the first surface to a second surface of the main body. The second surface includes at least one first hook and/or at least one second hook. The first hook is matched with a second hook of the main body of another sterile connection member to form a buckle connection, and/or the second hook is matched with a first hook of the main body of another sterile connection member to form a buckle connection. The sterile connection member further includes an unlocking hole formed in the main body and communicating with the buckle connection.

### FIELD

The present disclosure relates to the field of sterile connection technology, in particular to a sterile connection member and a sterile connection apparatus.

### BACKGROUND

Sterile connection technology is widely used in the biomedical field, such as cell culture, vaccine preparation, and biological reactions, etc., all of which require the establishment of sterile pathways for liquid transfer. Liquids are usually transported by plastic hoses from a first device or container to a second device or container, and this process requires the realization of aseptic docking between the piping of the first device and the piping of the second device. At this point, there is a need to connect the two docked lines by means of a sterile connection member.

In the existing technology, Patent Grant Publication No. CN107255198B discloses a sterile coupling arrangement formed by snapping together two structurally identical sterile coupling devices. A first side of the first coupling device is snapped to a second side of the second coupling device, and a second side of the first coupling device is snapped to a first side of the first coupling device. When the two are fastened together, they are connected in a non-removable manner, similar to two sickle-like structures hooked together, and if the two coupling devices are rigidly separated, the sickle-like structure will cause product waste and cost increase, and on the other hand will increase the working hours consumed.

In actual use, piping connections are usually involved, for example, piping 1 is connected to 1′, piping 2 is connected to 2′, piping 3 is connected to 3′, etc. However, due to the large number of piping, it is easy for misconnections to occur, for example, when the aseptic coupling device at the end of the piping 1 is connected with the aseptic coupling device at the end of the piping 2′, which results in the aseptic coupling arrangement being scrapped, and the need to re-connect the aseptic coupling device at the end of the piping. The aseptic coupling device at the end of the pipeline 2′ end is connected to the aseptic coupling device and snapped together again, which on the one hand results in a waste of the product and an increase in the cost, and on the other hand increases the consumption of working hours.

In order to solve the above problems in existing technology, there is an urgent need in this field for sterile connection apparatus with an anti-misoperation function, which, in the event of misconnection of the sterile connection member of the two ends of the transmission line, can amicably disengage the linkage relationship between the two sterile connection members without damaging the structure of the sterile connection member, and improve the service life of the sterile connection member.

### SUMMARY

A brief overview of one or more embodiments are provided below to provide a basic understanding of these embodiments. The summary is not an extensive overview of all of the embodiments that are contemplated, and is not intended to identify key or decisive elements in all embodiments. The sole purpose of the summary is to present some concepts of one or more embodiments in a simplified form as a prelude to the more detailed description that is presented later.

Embodiments of the present disclosure provide a sterile connection member including: a main body, a first surface of the main body is connected to an external pipeline. A fluid channel is arranged inside the main body. The fluid channel penetrates from the first surface to a second surface of the main body. The second surface includes at least one first hook and/or at least one second hook. The first hook is matched with a second hook of the main body of another sterile connection member to form a buckle connection, and/or the second hook is matched with a first hook of the main body of another sterile connection member to form a buckle connection. The sterile connection member further includes an unlocking hole formed in the main body and communicating with the buckle connection. The unlocking hole is configured for guiding an unlocking member to press the second hook and/or the first hook at the buckle connection to release the buckle connection. By using the sterile connectors with anti-misoperation functions mentioned above, in the event of a misconnection between the sterile connectors of the transmission pipeline at both ends, the connection relationship between the two sterile connectors can be amicably released without causing damage to the structure of the sterile connector, to improve its service life.

In some embodiments, the first side of the second surface includes the first hook, and the second side includes the second hook, the first hook cooperates with a second hook of another sterile connection member's main body to achieve a buckle connection, and the second hook cooperates with a first hook of another sterile connection member's main body to achieve a buckle connection.

Further, the first hook is an inner enclosure plate, and the second hook is a snap plate, the inner enclosure plate is provided with an inwardly extending snap strip at its end, the snap plate is provided with an outwardly extending snap hook at its end, the snap strip on the inner enclosure plate is connected to the snap hook on the snap plate of the another sterile connector for a first buckle connection, and the snap hook on the snap plate is connected to the snap strip on the inner enclosure plate of the another sterile connection member for a second buckle connection.

Even further, the second side of the second surface of the main body further includes an outer enclosure plate, the outer enclosure plate is provided on the outer side of the snap plate to limit the outward movement of the inner enclosure plate of the another sterile connection member in the buckle connection state.

In some embodiments, the sterile connection member further includes the unlocking member, the unlocking member laterally squeezes the second hook and/or the first hook at the buckle connection to cause it to undergo a restorable deformation in order to release the buckle connection.

Further, the unlocking member is provided with a barbed side edge at the end of the unlocking member, the width of the end of the side edge is greater than the width of an unlocking hole.

In some embodiments, in some embodiments, the sterile connection member further includes a film, the film is provided on a second side of the main body to cover an opening of the fluid channel on the second surface of the main body, an end of the film extends to the exterior of the main body for pulling and tearing away.

Further, the end of the film extending to the exterior of the main body is provided with a film puller by which the film is pulled away from the second surface of the main body, the film puller is detachably connected to the main body.

Even further, the film puller is provided with at least one bump and/or at least one recess on the outer side of the film puller, and the at least one bumps is snapped in correspondence with the at least one recess on the outer side of the film puller of the another sterile connection member, and/or the at least one recess is snapped in correspondence with the at least one bump on the outer side of the film puller of the another sterile connection member.

Even further, the film puller is provided with at least one bump on the inner side, the main body is provided with at least one recess on the outer wall, and the at least one bump on the inner side of the film puller cooperates with the at least one recess on the outer wall of the main body to realise the snap-fit of the film puller with the main body.

In some embodiments, a protective cover is also included, and the protective cover is detachably connected to the second surface of the main body.

Further, a first side of the inner side of the protective cover is provided with a splint, a gap exists between the splint and the edge of the protective cover, and the first hook extends into the gap to achieve an interference connection with the protective cover, and/or a second side of the inner side of the protective cover is provided with a holding frame, and the second hook of the main body extends into the holding frame to achieve an interference connection with the holding frame.

In one embodiment, the opening of the fluid channel on the second surface of the main body is provided with a sealing member, the end surface of the sealing member is higher than the end surface of the second side of the main body.

Another embodiment of the present disclosure also provides a sterile connection apparatus including at least one sterile connection member of any of the foregoing, and the first hook of the main body of the sterile connection member cooperates with the second hook of the main body of another sterile connection member to achieve a buckle connection, and/or the second hook of the sterile connection member cooperates with the first hook of the main body of another sterile connection member to achieve a buckle connection, the unlocking member of the sterile connection member is provided in the main body and connected to the buckle connection, for guiding the unlocking member to squeeze the second hook and/or the first hook at the buckle connection to release the buckle connection.

By using the above-described sterile connection apparatus having an anti-misoperation function, in the event of misconnection of the sterile connection member of the transmission line at both ends, it is possible to amicably disengage the coupling relationship between the two sterile connection members without causing damage to the structure of such sterile connection members, and to improve the service life of the sterile connection members.

### BRIEF DESCRIPTION OF THE DRAWINGS

The above embodiments of the present disclosure will be better understood after reading the detailed description of the embodiments of the present disclosure in conjunction with the following figures. In the figures, components are not necessarily drawn to scale, and components having similar related features may have the same or similar reference numerals.

FIG. 1 shows a schematic structure diagram of a sterile connection apparatus according to an embodiment of the present disclosure.

FIG. 2 shows a schematic structure diagram of the sterile connection member according to some embodiments of the present disclosure.

FIG. 3 shows a cross-sectional view of a sterile connection member according to some embodiments of the present disclosure.

FIG. 4 shows a sectional view of two sterile connections members connected to each other according to embodiments of the present disclosure.

FIG. 5 shows a second surface schematic of a sterile connection member according to some embodiments of the disclosure.

FIG. 6 shows the side front view of the film of the sterile connection member in the unfolded state as shown in FIG. 5.

FIG. 7A shows a front view of a film puller provided in accordance with some embodiments of the present disclosure;

FIG. 7B shows an inner schematic view of the film puller shown in FIG. 7A;

FIG. 7C shows an outer schematic view of the film puller shown in FIG. 7A;

FIG. 8A shows a schematic diagram of a structure in which two film pullers are snapped together, provided in accordance with some embodiments of the present disclosure;

FIG. 8B shows a side view of the two film pullers snapped together as shown in FIG. 8A;

FIG. 9 shows a schematic view of a structure in which a film puller is stripped in a sterile attachment device provided according to some embodiments of the present disclosure;

FIG. 10 shows a schematic view of the inner structure of a protective cover provided according to some embodiments of the present disclosure;

FIG. 11 shows a schematic structural diagram of the protective cover shown in FIG. 10 being capped with the aseptic connection member.

Reference signs

| | |
|---|---|
| 10 | sterile connection apparatus; |
| 100 | first sterile connection member; |
| 200 | second sterile connection member; |
| 110, 210 | main body; |
| 111, 211 | first side; |
| 112, 212 | second side; |
| 1110, 2110 | inner enclosure plate; |
| 1111, 2111 | snap strip; |
| 1120, 2120 | snap plate; |
| 1121, 2121 | hook; |
| 113, 213 | first side of the main body; |
| 114, 214 | second side of the main body; |
| a | first step; |
| a′ | second step; |
| C | first slot; |
| C′ | second slot; |
| 1122, 2122 | outer enclosure plate; |
| D | inner enclosure space; |
| 120 | extension joint; |
| 121 | barbed structure |
| 122, 222 | fluid channel; |
| 130, 230 | sealing member; |
| 140, 240 | film; |
| 1401, 2401 | first side of the film; |
| 1402, 2402 | second side of the film; |
| 141 | connection position; |
| 142 | connection area; |
| 150, 250 | film puller; |
| 151, 251 | inner side bump; |
| 1131 | snap recesses; |
| 152, 252 | outer side bump; |
| 153, 253 | outer side recess; |
| 160, 260 | unlocking hole |
| 161, 261 | unlocking member; |
| 170, 270 | protective cover; |
| 171 | splint; |
| 172 | splint gap |
| 173 | holding frame; |
| 174 | through-hole; |
| 175 | grip section; |
| A | first surface of the main body; |
| B | second surface of the main body. |

### DETAILED DESCRIPTION OF EMBODIMENTS

The embodiments of the present disclosure are described in the following detailed description. Other embodiments of the present disclosure will be readily apparent from this disclosure. Although the description of the present disclosure will be described in conjunction with the embodiments, this is not a limitation of the present disclosure. On the contrary, the disclosure is described in connection with the embodiments to cover other alternatives or modifications that are possible in the embodiments of the present disclosure. In order to provide a thorough understanding of the present disclosure, many specific details are included in the following description. The present disclosure may also be practiced without these details. In addition, some specific details are omitted in the description in order to avoid confusing or obscuring the present disclosure.

In the description of the present disclosure, it should be noted that the terms “installation”, “connecting”, and “connected” should be understood broadly unless explicitly stated and defined otherwise. For example, the terms “installation”, “connecting”, and “connected” may be either a fixed connection, a detachable connection, or an integral connection; the terms may be either a mechanical connection or an electrical connection; the terms also may be either a direct connection, an indirect connection through an intermediate medium, or an internal connection between two components. The specific meaning of the above terms in the present disclosure can be understood in a specific case.

In addition, “up”, “down”, “left”, “right”, “top”, “bottom”, “horizontal”, “vertical” used in the following description shall be understood as the orientation described in the paragraph and shown in the related figure. The relative term is configured for convenience of description only, and does not mean that the device described therein is to be manufactured or operated in the specific orientation, and therefore should not be construed as limiting the present disclosure.

Understandably, although the terms “first”, “second”, “third”, etc. may be used to describe various components, regions, layers and/or portions to distinguish different components, regions, layers and/or portions, the order of these components, regions, layers and/or portions described above should not be limited by the terms. Therefore, a first component, region, layer and/or portion mentioned below may be also mentioned as a second component, region, layer and/or portion without departing from some embodiments of the present disclosure.

As mentioned above, there is a patented technology, the Patent Grant Publication No. CN107255198B, which discloses a sterile coupling arrangement consisting of two structurally identical sterile coupling devices. When the two are fastened together, they are connected in a non-removable manner, similar to two sickle-like structures hooked together, and if the two coupling devices are rigidly separated, the sickle-like structure will cause destructive damage to the sickle-like structure, rendering the two coupling devices incapable of continuing to be used. For example, the sterile coupling arrangement at the end of pipeline 1 is connected with the sterile coupling arrangement at the end of pipeline 2′, resulting in the abolition of the sterile coupling arrangement, and it is necessary to reconnect the sterile connection apparatus at the end of pipeline and then connect the connection, which on the one hand will cause product waste and cost increase, and on the other hand will increase the working hours consumed.

In order to solve the above problems in the prior art, the present disclosure provides a sterile connection member and a sterile connection apparatus, by using the above the sterile connection member with an anti-misoperation function, in the event of misconnection of the sterile connection member of the two ends of the transmission line, can amicably disengage the linkage relationship between the two sterile connection members without damaging the structure of the sterile connection member, and improve the service life of the sterile connection member.

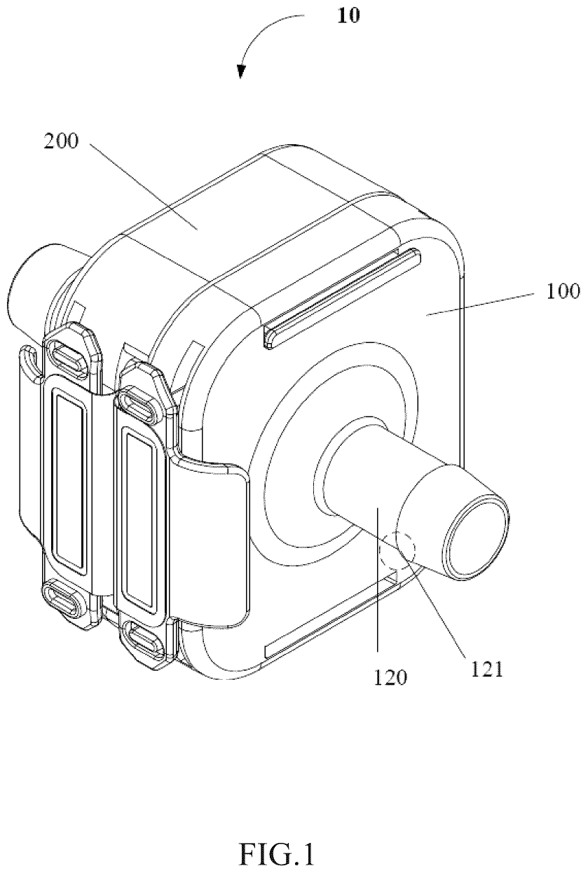

The sterile connection apparatus provided in another embodiment of the present disclosure may include sterile connection members provided in one embodiment of the present disclosure. Referring first to FIG. 1, FIG. 1 shows a schematic structure diagram of a sterile connection apparatus according to an embodiment of the present disclosure.

In some non-limiting embodiments of the present disclosure, the sterile connection apparatus 10 includes the first sterile connection member 100 and the second sterile connection member 200. The sterile connection apparatus 10 will be described below in conjunction with the first and second sterile connection members 100, 200.

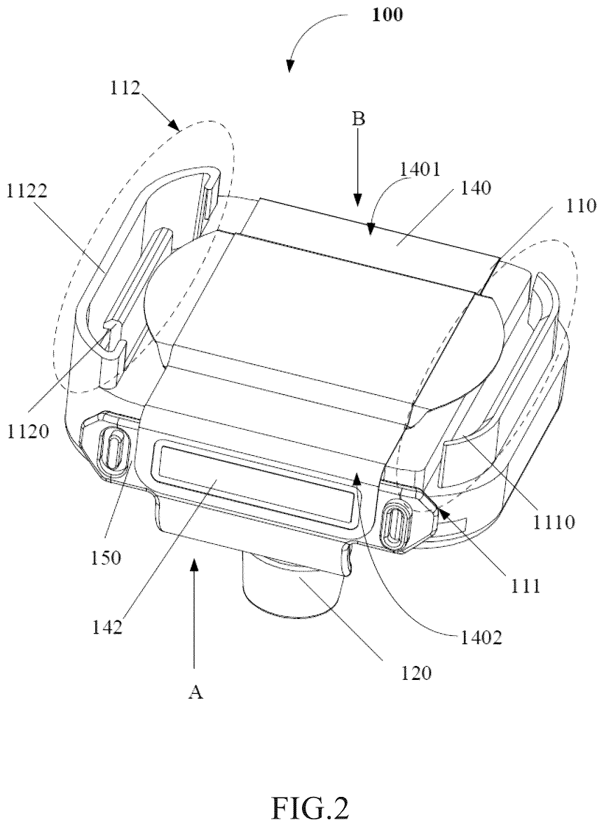

Please refer to FIG. 2, FIG. 2 shows a schematic structure diagram of the sterile connection member according to some embodiments of the present disclosure.

In some embodiments, the first surface A of the main body 110 may be directly connected to an external line.

In the embodiment combining FIGS. 1 and 2, the first surface A of the main body 110 of the first sterile connection member 100 in the sterile connection apparatus 10 may be provided with an extension joint 120 for mating connection with an external line. The external piping herein is a broad concept, and may include narrowly defined piping for connecting to a device or container, or may include the device or container itself, i.e., the extension joint 120 is directly connected to the device or container.

In one embodiment, the outer wall of the extension joint 120 may be provided with a barbed structure 121 for increasing the friction between the extension joint 120 and the external plastic hose, improving the sealing performance during connection, and preventing slippage between the extension joint 120 and the plastic hose.

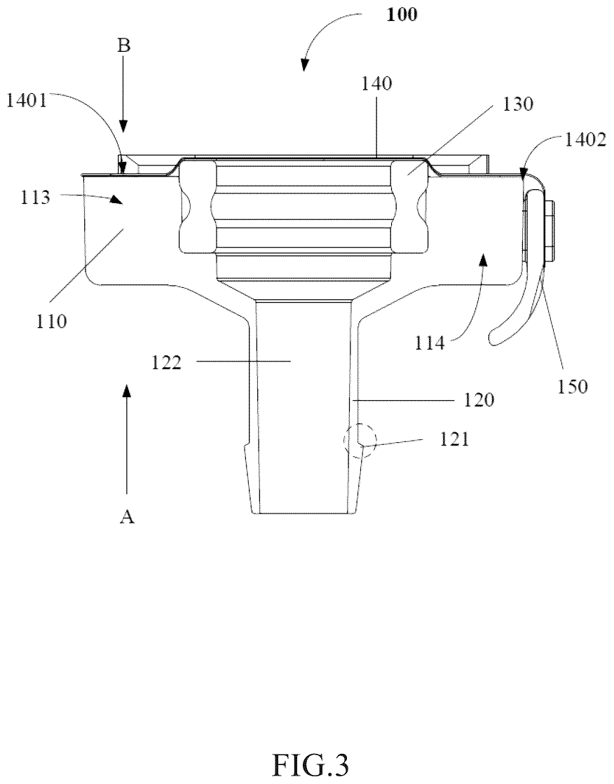

Please refer to FIG. 3, FIG. 3 shows a cross-sectional view of a sterile connection member according to some embodiments of the present disclosure. As shown in FIG. 3, the interior of the extension joint 120 is constructed with a fluid channel 122 that runs through a first surface A and a second surface B of the main body 110. A sealing member 130 is provided at an opening of the fluid channel 122 on the second surface B of the main body 110. A step groove is provided in the interior wall of the end of the fluid channel 122 that is remote from the extension joint 120, and the sealing member 130 may be provided in the step groove. The sealing member 130 may, but is not limited to, be a rubber ring, a silicone ring. In one embodiment, the end surface of the sealing member 130 is higher than the end surface of the main body 110, ensuring a good sealing when the two sterile connection members 100, 200 are butted together and the end surfaces of the two sealing members 130, 230 are extruded together.

In some embodiments of the present disclosure, the second surface B of the main body 110 of the first sterile connection member 100 and the second surface B of the main body 210 of the second sterile connection member 200 in the sterile connection apparatus 10 are cooperatively connected.

In one embodiment, the second surface of the body of the sterile connection member may include at least one first hook and/or at least one second hook. The first hook of the main body of the sterile connection member cooperates with the second hook of the main body of the another sterile connection member to achieve a buckle connection, and/or the second snap hook of the main body of the sterile connection member cooperates with the first hook of the main body of the another sterile connection member to achieve a buckle connection.

In some embodiments, the first hook may be an inner flange and the second hook may be a snap plate to facilitate a clearer understanding of such first hook and second hook. In the following, the embodiments in which the inner flange is the first hook and the snap plate is the second hook will be used to explain the sterile connection members and sterile connection apparatuses protected by the present disclosure.

Please continue to return to FIG. 2. In the embodiment shown in FIG. 2, the first side 111 of the second surface B of the main body 110 of the first sterile connection member 100 includes an inner enclosure plate 1110 disposed vertically on that end face, and the second side 112 of the second surface B includes a snap plate 1120 disposed vertically on that end face. The first sterile connection member 100 connects to the second sterile connection member 100 by means of the inner enclosure plate 1110 and the snap plate 1120 on the second surface B of its main body 110 to achieve a mating connection with the second sterile connection member 200.

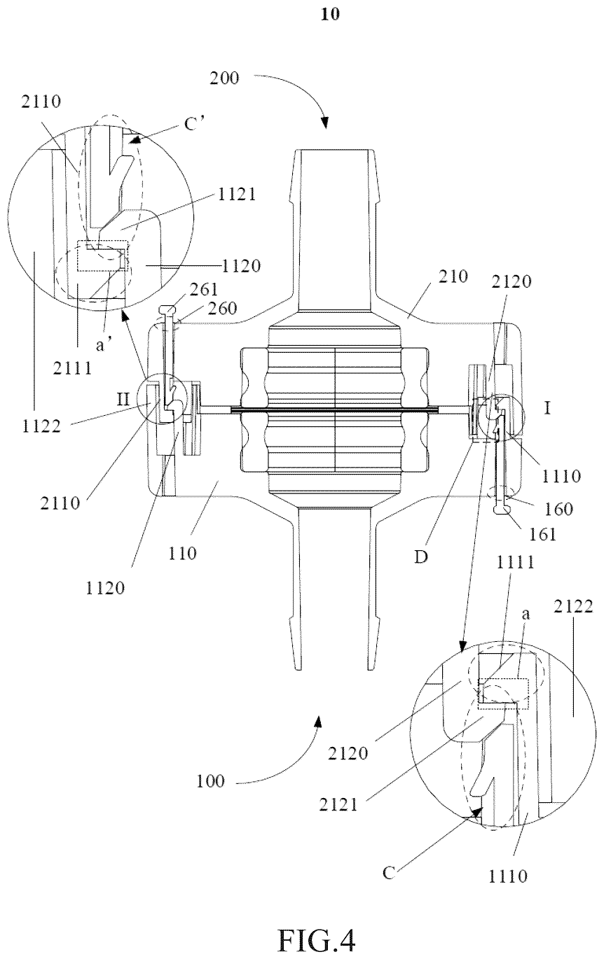

Referring specifically to FIG. 4, FIG. 4 shows a sectional view of two sterile connections members connected to each other according to embodiments of the present disclosure.

In the embodiment shown in FIG. 4, in a locally enlarged region I, the inner enclosure plate 1110 of the first side 111 of the main body 110 of the first sterile connection member 100 cooperates with the snap plate 2120 of the second side 212 of the main body 210 of the second sterile connection member 200 to realize a buckle connection. In partially enlarged region II, the inner enclosure plate 2110 of the first side of the main body 210 of the second sterile connection member 200 cooperates with the snap plate 1120 of the second side 112 of the main body 110 of the first sterile connection member 100 to realize the buckle connection.

Further, referring to the partially enlarged view in the region I, the end of the inner enclosure plate 1110 of the first sterile connection member 100 is provided with an inwardly extending snap strip 1111, which may be provided in the form of a step. On the inner side of the inner enclosure plate 1110, a first snap slot C is formed between the snap strip 1111 and a vertical wall of the inner enclosure plate 1110. An end portion of the snap plate 2120 of the second sterile connection member 200 is provided with an outwardly extending hook 2121. The inner sides of the snap strip 1111 on the inner enclosure plate 1110 of the first sterile connection member 100 and the hook 2121 on the snap plate 2120 of the second sterile connection member 200 contact each other to realize a first buckle connection, i.e., the hook 2121 of the snap plate 2120 is just on the first step ‘a’ position within the first slot C of the inner enclosure plate 1110.

Similarly, referring to the partially enlarged view in region II, the end of the inner enclosure plate 2110 of the second sterile connection member 200 is provided with an inwardly extending snap strip 2111, which may be provided in the form of a step. A second slot C′ is formed between this snap strip 2111 and a vertical wall of the inner enclosure plate 2110 on the inner side of the inner enclosure plate 2110. The end of the snap plate 1120 of the first sterile connection member 100 is provided with a snap hook 1121 extending outwardly. The inner sides of the snap strip 2111 on the inner enclosure plate 2110 of the second sterile connection member 200 and the card hook 1121 on the snap plate 1120 of the first sterile connection member 100 are in contact with each other for realizing the second buckle connection, i.e., the snap hook 1121 of the snap plate 1120 is just caught into the second slot C′ of the inner enclosure plate 2110 at the position of the second step a′.

The above-described buckle connection, i.e., the first and second double buckle connection, is realized for both sides of the first and second sterile connections members 100, 200 of the sterile connection apparatus 10, which can make the buckle connection between the first and second sterile connectors 100, 200 of the sterile connection apparatus 10 more tight.

As can be seen from the above-described snap-fit method of the sterile connection members 100, 200, the sterile connection apparatus 10 provided in the present disclosure for connecting for fluid transfer can be snap-fit connected by two sterile connection members with identical structures, i.e. the structures of the sterile connection members as the transmitting side and the receiving side can be identical. The as-transmitter and receiver sterile connection members in the sterile connection apparatus 10 can be gender-neutral and yin-yang-neutral, which makes the production and manufacture of the sterile connection apparatus 10 simpler, and only one set of molds is needed to produce sterile connection members to be assembled to obtain the sterile connection apparatus 10.

In another embodiment of the present disclosure, the sterile connection apparatus may also include two structurally different sterile connections (not shown in the accompanying drawings). For example, both the first side and the second side of the second face of the main body of the first sterile connection member may be provided with an inner enclosure plate perpendicular to that end face. Correspondingly, the first side and the second side of the second face of the main body of the second sterile connection member may both be provided with a snap plate perpendicular to that end face. Both of the inner enclosure plates in the first sterile connection member are provided with inwardly extending snap strip at the end portions. The end portions of both of the snap plates in the second sterile connection member are provided with snap hooks extending outwardly. The two inner enclosure plates in the first sterile connection member and the two snap plates in the second sterile connection member can still realize the first and second double buckle connections.

Further, please combine FIGS. 2 and 4, the second side 112 of the second surface B of the main body 110 of the first sterile connection member 100 may also include an outer enclosure plate 1122. The outer enclosure plate 1122 is provided on the outer side of the snap plate 1120, which is also on the second side 112, for restricting the inner enclosure plate 2110 of the second sterile connection member 200, which is in the buckle connection state with the snap plate 1120 of the first sterile connection member 100 from moving outwardly.

In the partially enlarged view II of FIG. 4, when the first sterile connection member 100 and the second sterile connection member 200 are in buckle connection, the inner enclosure plate 2110 of the second sterile connection member 200 is just inserted between the outer enclosure plate 1122 and the snap plate 1120 of the first sterile connection member 100, to realize a second buckle connection with the snap plate 1120 of the first sterile connection member 100. Due to the limiting effect of the outer enclosure plate 1122, the inner enclosure plate 2110 of the second sterile connection member 200 in the buckle connection state is unable to undergo a large displacement in the transverse direction, and the problem of loosening of this buckle connection part can be avoided.

In the partially enlarged view I of FIG. 4, the outer enclosure plate 2122 of the second sterile connection member 200 also plays the same role, which will not be repeated here.

Unlocking holes are also provided in the sterile connectors provided by the present disclosure. In one embodiment, the interiors of the first sterile connection member 100 and the second sterile connection member 200 include unlocking holes 160, 260, respectively, as shown in FIG. 4.

In the case of the first sterile connection member 100, for example, the opening of the unlocking hole 160 is located on the first surface A of the main body 110, and the unlocking hole 160 extends from this first surface A to the buckle connection position between the main body 110 and the second sterile connection member 200, for guiding the unlocking member 161 to reach into, contact, and transversely squeeze the end face of the snap plate 2120 of the main body 210 of the second sterile connection member 200 and/or the inner enclosure plate 1110 of the first sterile connection member 100 itself to unlock the buckle connection between the two sterile connection members 100, 200.

In one embodiment, the unlocking member 161 may be moved back and forth within the first slot C, in a direction perpendicular to the end face of the main body 110 of the first sterile connection member 100, including the movement of reaching into and pulling away from the buckle connection position in the unlocking hole 160. Pushing the unlocking member 161 may cause it to reach into the buckle connection location of the snap hook 2121 of the snap plate 2120 of the second sterile connection member 200 and the snap strip 1111 of the inner enclosure plate 1110 of the first sterile connection member 100. The unlocking member 161 may force the snap strip 1111 of this inner enclosure plate 1110 and/or the snap hook 2121 of the snap plate 2120 to deform and release the buckle connection between the two.

Further, the ability of the snap strips 1111 on the inner enclosure plate 1110 and/or the snap hooks 2121 on the snap plate 2120 of the two sterile connection members 100, 200 to undergo a recoverable deformation is primarily accomplished by a combination of manufacturing materials and structures, for example, the materials of the sterile connection members 100, 200 may be polycarbonate (PC) and Polyarylate (PAR), for example.

In the process of continuously pushing the unlocking member 161, the unlocking member 161 extrudes the inner enclosure plate 1110 and the snap plate 2120 transversely, and the ends of the inner enclosure plate 1110 of the sterile connection member 100 and the snap plate 2120 of the other sterile connection member 200 undergo a transverse deformation due to the extrusion of the unlocking member 161. In one embodiment, this includes that the unlocking member 161 squeezes the snap hook 2121 originally resting on the first step “a” of the snap strip 1111 of the inner enclosure plate 1110, causing it to detach from the first step ‘a’ and fall into a step lower than the first step ‘a’, to release the buckle connection between the inner enclosure plate 1110 and the snap plate 2120.

However, after the buckle connection relationship between the two is released, since the above-described operation of separating the two sterile connection members 100, 200, which are snap connected together, by means of the unlocking member 161 does not produce structural damage thereto, the snap plate 2120 of the second sterile connection member 200 and the inner enclosure plate 1110 of the first sterile connection member 100 can be deformed and recovered, and the two sterile connection members 100, 200 still may continue to be properly connected.

In some embodiments, continuing with the example of the first sterile connection member 100, an inner enclosure space D may also be provided between the inner enclosure plate 1110 and the centre of the main body 110. The inner enclosure space D may provide a space for the snap plate 2120, which has been extruded, to be displaced inwardly, and the snap plate 2120, which has been extruded by the unlocking member 161, may be deformed, and be displaced transversally in the inner enclosure space D, in a direction relatively far away from the outer enclosure plate 2122, to release the buckle connection with the inner enclosure plate 1110 of the first sterile connection member 100.

For the structural design of the unlocking member 161, in some embodiments, as shown in the partially enlarged view I in FIG. 4, the head end of the portion of the unlocking member 161 that extends into the buckle connection position between the snap plate 2120 and the inner enclosure plate 1110, i.e., the unlocking head of the unlocking member 161 may be a wedge-shaped end, and the snap hook 2121 of the snap plate 2120 is also a wedge-shaped end, and a contact surface of the unlocking member 161 and the snap hook 2121 is also wedge-shaped, facilitating the unlocking member 161 to reach into the buckle connection position and laterally squeeze the snap plate 2120 inwardly for snap unlocking.

The tail end of the portion of the unlocking member 161 that extends into the buckle connection position of the snap plate 2120 and the inner enclosure plate 1110, i.e., the unlocking head of this unlocking member 161 may be provided with a barb-like side edge, which barb-like side edge can be deformed slightly and recovered. When the unlocking member 161 is inserted into the unlocking hole 160, the barb-like side edge undergoes micro-deformation in a direction in which the side edge is brought closer to the main body of the unlocking member 161 to facilitate the unlocking member 161 to smoothly extend into the unlocking hole 160. When the unlocking member 161 is extended to the point where it is about to contact the buckle connection position, the unlocking head of the unlocking member 161 enters into the inner enclosure space D on the inner side of the inner enclosure plate 1110, and at this time, the barbed side edge resumes its elastic deformation. After the deformation is restored, the width of the end of the unlocking head with the side edge of the unlocking member 161 is greater than the width of the unlocking hole 160, ensuring that the unlocking member 161 cannot be withdrawn from the unlocking hole 160 after the unlocking member 161 is in the state of restoring the deformation, and thus preventing the unlocking member 161 from detaching from the main body 110 of the first sterile connection member 100.

The end of the unlocking member 161 extends beyond the unlocking hole 160 and is exposed outside of the main body 110 for use by an operator to manipulate the application of force.

The structure and unlocking principle of the unlocking hole 260 and the unlocking member 261 of the second sterile connection 200, which is matingly connected to the first sterile connection 100, are the same as those of the unlocking hole 160 and the unlocking member 161 in the first sterile connection 100, and will not be repeated herein.

In one embodiment of the present disclosure, of the two structurally different sterile connection members in the sterile connection apparatus, it may be present that only one of the sterile connection members includes an unlocking hole. The first surface of the first sterile connection member including two inner enclosure plates may be provided with two unlocking holes, and the first surface of the second sterile connection member including two snap plates is not provided with unlocking holes.

The two unlocking members may correspondingly extend into the two unlocking holes of the first face of the first sterile connection member and squeeze the two snap plates of the second sterile connection member at the two buckle connection locations to release the buckle connection between the outer enclosure plates of the first sterile connection member and the snap plates of the second sterile connection member.

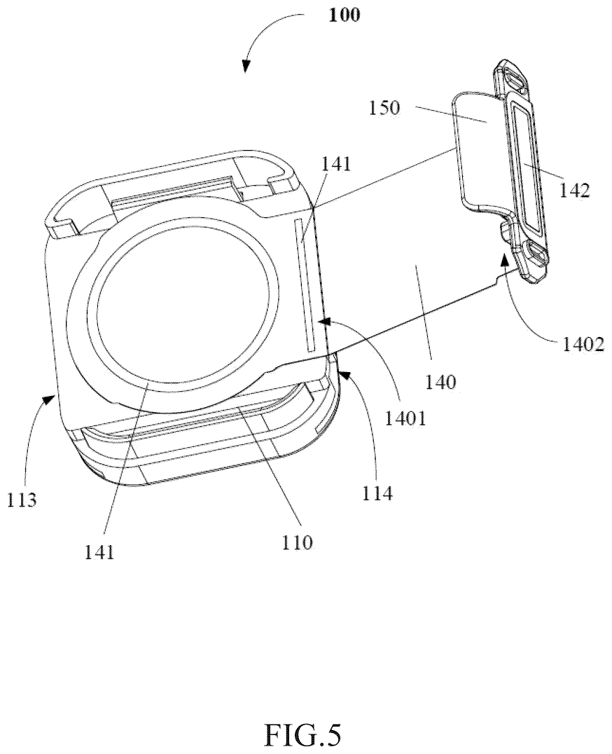

Referring on to FIG. 5, FIG. 5 illustrates a schematic view of the second surface of a sterile connector provided according to some embodiments of the present disclosure.

It can be referred to together with FIGS. 2, 3 and 5, as an example of the first sterile connection member 100, the fluid channel 122 within the extension joint 120 is also provided with a film 140 for water and bacteria barrier at the opening of the second surface B of the main body 110. The material of the film 140 can be polyethylene, polyethersulfone, polytetrafluoroethylene, and the like. When the sterile connection apparatus 10 is configured, the film 140, 240 between the bodies 110, 210 of the two sterile connections 100, 200 that are snapped together can be pulled out.

In some embodiments, the first sides 1401, 2401 of the films 140, 240 are connected to the first side faces 113, 213 of the main bodies 110, 210, and the second sides 1402, 2402 of the films 140, 240 are connected to the second side faces 114, 214 of the main bodies 110, 210, to allow the films 140, 240 to cover each of openings of the internal fluid channels 122, 222 of the two sterile connection members 100, 200 on the second surface B of their respective main bodies 110, 210. In one embodiment, the first side faces 113, 213 and the second side faces 114, 214 of the main bodies 110, 210 are located on opposite sides of the main bodies 110, 210, respectively.

In one embodiment, the first side 1401, 2401 of the film 140, 240 or the second side 1402, 2402 of the film 140, 240 may be provided with a film pull member 150, 250. The film 140, 240 may be snapped to the first side face 113, 213 or the second side face 114, 214 of the main body 110, 210 by this film puller 150, 250.

Referring specifically to FIGS. 2 and 3, which illustrate the first sterile connection 100 with the film 140 in a folded state, and FIG. 5 illustrates the first sterile connection 100 with the film 140 in an unfolded state.

A first side 1401 of the film 140 is connected to a first side face 113 of the main body 110, and a second side 1402 of the film 140 is provided with a film puller 150. The film 140 is folded in a double-layer structure and is snapped to the first side face 113 of the main body 110 via the film puller 150. In one embodiment, the first side face 113 and the second side face 114 of the main body 110 are located on opposite sides of the main body 110, which may be, for example, the left and right sides of the main body 110 in FIG. 5. The film 140 and the main body 110 may be connected together and the film 140 and the film puller 150 may be connected together by a process including, but not limited to, a heat fusion welding process, for example, by gluing or the like, which may also achieve a connection effect.

In one embodiment, as shown in FIG. 2 or as shown in FIG. 5, the connection region 142 between the second side 1402 of the film 140 and the film puller 150 can be used to set a pattern shape, which specifically can include but is not limited to being a name, an icon, or a logo representing a company. In some embodiments of the present disclosure, the film 140 and the film puller 150 can have color difference, and the welding head of the hot melt welding machine is set to the desired pattern shape, and a patterned connecting region 142 is formed after the welding is completed.

The transverse width between the first side 1401 and the second side 1402 of the film 140 is large to completely cover the sealing member 130 and has a margin at its end to extend to the exterior of the main body 110 for pulling and tearing away from the main body 110.

In order to better cover the film 140 over the opening of the sealing member 130, at least one connecting position 141 may be provided at the first side 1401 of the film 140 and the outer periphery of the sealing member 130 of the main body 110 to secure the film 140 over the main body 110.

In one embodiment, as shown in FIG. 5, connection bits 141 may be provided near a location where the film 140 is folded to cover the end face of the main body 110 and/or an annular connection position 141 may be provided at the outer periphery of the sealing member 130, and the inner diameter of the annular connection position 141 is greater than the outer diameter of the sealing member 130.

Further, in some embodiments, the film 140 for covering the sealing member 130 is folded, and the folded lower film 140 is configured to secure the sealing member 130 with the connection position 141 at the outer periphery of the sealing member 130, and the folded upper film 140 is configured in a manner that the film 140 rolls on itself to facilitate tearing away from the main body 110 when the film 140 is pulled.

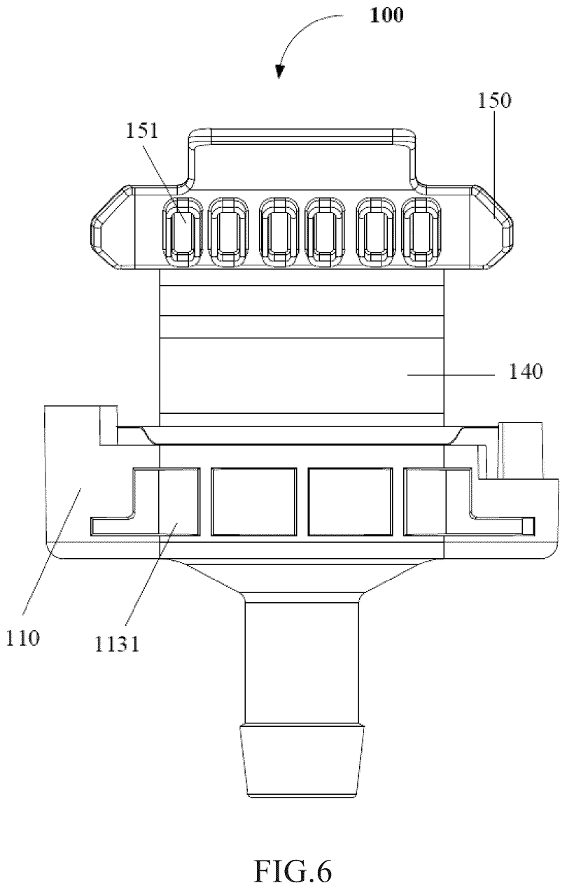

Please refer to FIG. 6, FIG. 6 illustrates a side front view of the film of the sterile connector shown in FIG. 5 in an unfolded state.

As shown in FIG. 6, the second side 1402 of the film 140 connects the film puller 150 to the first side face 113 of the main body 110 to achieve a snap fit. The inner side of the film puller 150 may be provided with inner side bumps 151. The first side face 113 of the main body 110 is provided with snap recesses 1131 corresponding to the positions of the inner side bumps 151 of the film puller 150. In one embodiment, according to actual needs, the number of snap recesses 1131 on the main body 110 may correspond to the inner side bumps 151 of the film puller 150, and may also not correspond to the number of inner side bumps 151 as shown in FIG. 6. It is sufficient to satisfy that the positions of the snap recesses 1131 correspond to the positions of the inner side bumps 151, i.e., the inner side bumps 151 of the film puller 150 can be inserted exactly and correspondingly into the snap recesses 1131 on the main body 110 to complete the snap.

The above-mentioned detachable connection between the film puller 150 and the main body 110 in the form of the bumps and recesses, as well as the design structure of the bumps and recesses, is only a non-limiting embodiment provided by the present disclosure, which is intended to clearly demonstrate the main idea of the present disclosure and to provide a specific solution for easy implementation by the public, and is not intended for limiting the scope of protection of the present disclosure. The user may, according to the actual needs, choose other removable methods, such as pasting, etc., that meet the connection needs and the firmness of the connection.

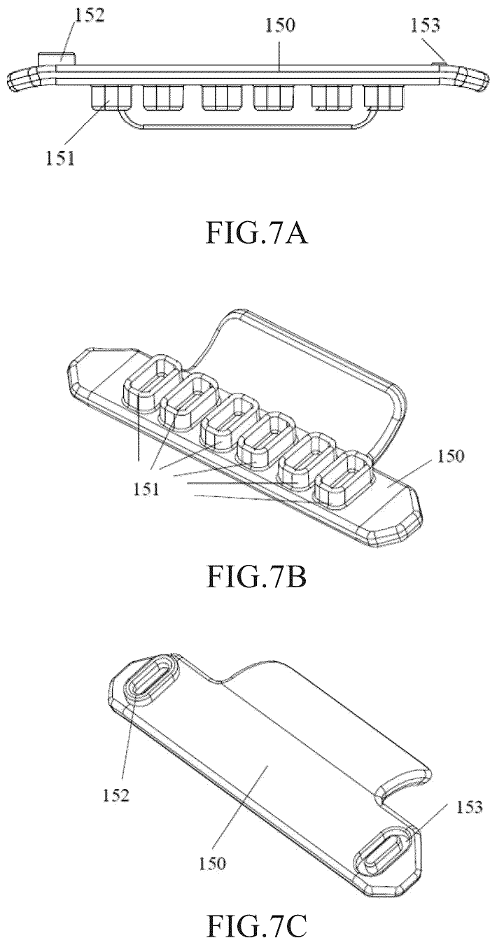

The specific structure of the film puller 150 can be referred to FIGS. 7A˜FIG. 7C, FIG. 7A illustrates a front view of the film puller provided according to some embodiments of the present disclosure, FIG. 7B illustrates an inner schematic view of the film puller shown in FIG. 7A, and FIG. 7C illustrates an outer schematic view of the film puller shown in FIG. 7A.

In conjunction with FIGS. 7A to 7C, the inner side of the film puller 150 has at least one inner side bump 151, and may include inner side bumps 151 to be snapped in correspondence with the snap recess 1131 of the main body 110. The outer side of the film puller 150 may be provided with at least one outer side bump 152 or at least one outer side recess 153. The outer tab structure and the corresponding outer recess structure may be snapped together.

For example, the at least one outer side bump 152 of the first sterile connection 100 film puller 150 may be snapped in correspondence with the at least one recess 253 on the outer side of the film puller 250 of the second sterile connection 200 of the same construction as it or the at least one outer side recess 153 of the first sterile connection 100 film puller 150 may be snapped in correspondence with the at least one recess 153 on the outer side of the second sterile connection 200 of the same construction as it.

In one embodiment, the outer side of the film puller 150 may also include both the at least one outer side bump 152 and the at least one outer side recess 153. The film puller 250 of the second sterile connection member 200 of the same construction as it includes a correspondingly equal number of the at least one outer side recess 253 and the at least one outer side bump 252 to be snapped in correspondence with the outer side of the film puller 150 of the first sterile connection member 100.

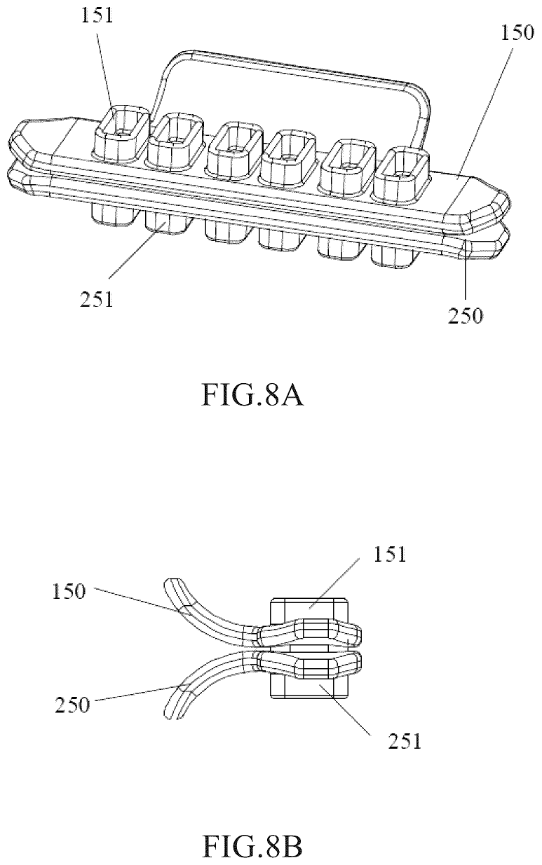

Specific reference may be made to FIG. 8A and FIG. 8B, where FIG. 8A illustrates a schematic structural view of two film pullers snapped together according to some embodiments of the present disclosure, and FIG. 8B illustrates a side view of the two film pullers snapped together shown in FIG. 8A.

The aforementioned external connection structures between the two film pullers 150, 250 include, but are not limited to, the bump and recess connection structures mentioned in the above embodiments.

In some embodiments provided by the present disclosure, the film 140 may be driven away from the end face of the main body 110, 210 by applying a pulling force to the film puller 150, 250 connecting the films 140, 240 at the openings of the respective seals 130, 230, prior to the use of the sterile connection members 100, 200 for connecting the fluids.

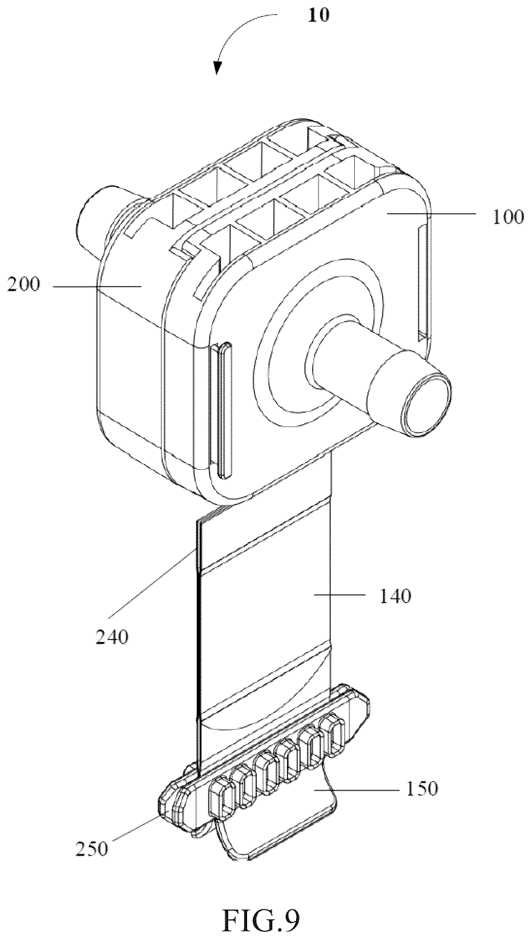

FIG. 9 illustrates a schematic view of a structure in which a film puller is stripped in a sterile connection apparatus provided according to some embodiments of the present disclosure. As shown in FIG. 9, the first sterile connection member 100 in the sterile connection apparatus 10 is matingly connected to a second sterile connection member 200 for conduction of fluids in the two devices, containers. The outer side between the film puller 150 of the first sterile connection 100 and the film puller 250 of the second sterile connection 200 are connected to each other by a connecting structure, which connects the two film pullers 150, 250 together and applies a pulling force to them at the same time. Since the film pullers 150, 250 are connected to respective films 140, 240, the films 140, 240 roll away from the corresponding main bodies 110, 210 to tear away when the film pullers 150, 250 are pulled. By connecting the two film pulling members 150, 250 together and applying force at the same time, it can be ensured that the two films 140, 240 are peeled off in synchronization, to avoid unsynchronized peeling leading to contamination of the seals 130, 230 with stray bacteria.

In one embodiment, a protective cover may be provided on the side of the sterile connectors 100, 200 provided by the present disclosure that has the films 140, 240 folded over. The protective cover may be removably connected to the main body 110, 210 of the sterile connectors 100, 200.

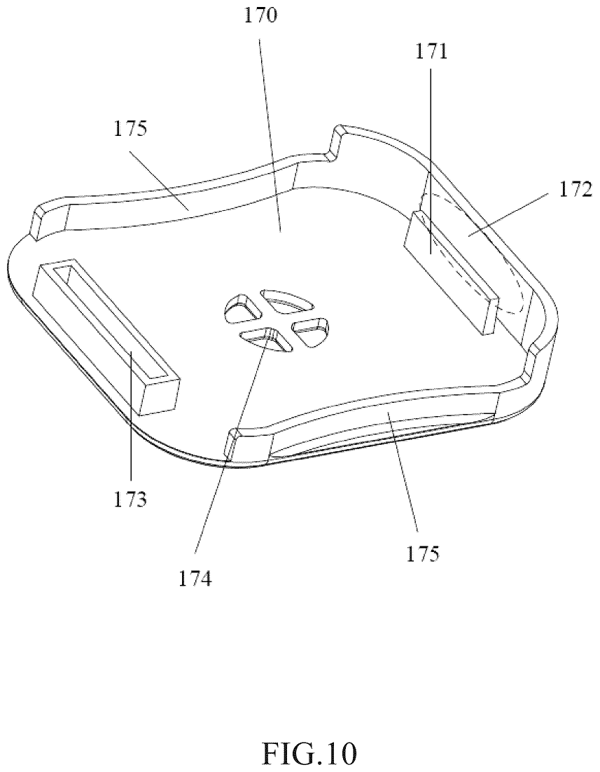

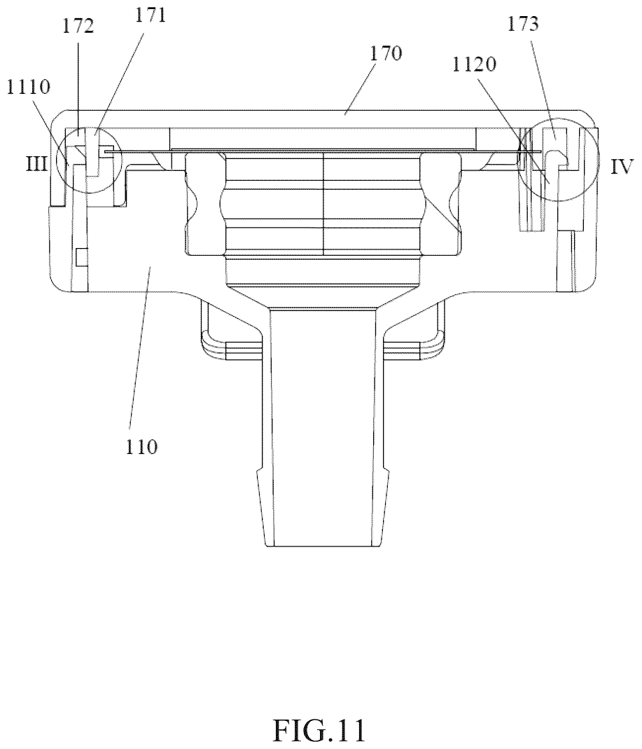

Please refer to FIG. 10 and FIG. 11, FIG. 10 illustrates a schematic diagram of the inner structure of the protective cover provided in accordance with some embodiments of the present disclosure, and FIG. 11 illustrates a schematic diagram of the structure of the protective cover shown in FIG. 10 when it is capped with the sterile connection member.

As shown in conjunction with FIGS. 10 and 11, the first side of the inner side of the protective cover 170 may be provided with a splint 171 having a splint gap 172 between the splint 171 and the edges of the protective cover 170. Continuing with the example of the first sterile connection member 100, the main body 110 of the first sterile connection member 100 is provided with the inner enclosure plate 1110 and a snap plate 1120.

In one embodiment, referring to the portion of region III shown in FIG. 11, the inner enclosure plate 1110 of the main body 110 extends into the splint gap 172 of the protective cover 170 when the protective cover 170 is capped with the first sterile connection member 100, and the inner enclosure plate 1110 and the splint gap 172 achieve an interference fit to be connected.

In one embodiment, the second side of the inner side of the protective cover 171 may be provided with a holding frame 173. Referring to part IV of the region shown in FIG. 11, when the protective cover 170 is capped with the first sterile connection member 100, the snap plate 1120 on the main body 110 extends into the holding frame 173 on the inner side of the protective cover 170, and realizes an interference fit with the holding frame 173 to connect.

The above-described connection between the protective cover 170 and the main body 110 is only a non-limiting embodiment provided by the present disclosure, which is intended to clearly demonstrate the main idea of the present disclosure and to provide a specific solution that is easy to be implemented by the public, and is not intended for limiting the scope of protection of the present disclosure. The user may choose other detachable methods that meet the connection needs and the firmness of the connection according to the actual needs.

Please continue to refer to FIG. 10, in one embodiment, the protective cover 170 may also be provided with a through-hole 174, which enables the gas to enter into the cover when this first sterile connection member 110 is in use to contact the structures such as the film 140 and the main body 110, and to realize the purpose of gas sterilization. For example, the sterile connectors are used in scenarios where ethylene oxide gas sterilization is employed.

In one embodiment, the protective cover 170 may also be provided with a grip portion 175 that facilitates hand grip for easy assembly and removal of the protective cover 170. The grip portion 175 may be provided as a recessed or convex rib structure on the edge of the protective cover 170. Similarly, the above-described structure of the protective cover 170 grip portion 175 is only a non-limiting embodiment provided by the present disclosure, and the manner in which the grip portion 175 is provided is not limited thereto.

By means of the above-described sterile connection members 100, 200 constitute the sterile connection apparatus 10 protected by another embodiment of the present disclosure. The sterile connection apparatus 10 is assembled and used as follows: the two sterile connection members 100, 200 are taken out, the protective covers 170, 270 of both of them are removed, and by means of the buckle connection between the snap plates 1120, 2120 and the inner enclosure plates 2110, 1110, the main bodies 110, 210 of the two sterile connection members 100, 200 are connected together. In case of misoperation, the two sterile connection members 100, 200 can be separated by simultaneously pressing the two locking release members 161, 261, and then a subsequent correct connection operation can be performed.

After correct connection, the two film puller 150, 250 are removed from the corresponding main bodies 110, 210 and connected by the fit of the recesses to the bumps. Simultaneous application of a tensile force to the two film puller 150, 250 peels off the two films 140, 240, and the seals 130, 230 of the two sterile connection members 100, 200 achieve sterile docking. Thus, the piping of the first device or container and the piping of the second device or container achieve sterile connection through this sterile coupling apparatus 10.

In summary, the present disclosure provides the sterile connection member and the sterile connection apparatus, which, by using the above-mentioned sterile connection member having an anti-misoperation function, can amicably release the coupling relationship between the two sterile connections members in the event of a misconnection of the sterile connections members of the transmission lines at both ends without causing damage to the structure of the sterile connection member, and improve the service life of the sterile connection member.

Further, the sterile connection member and the sterile connection apparatus provided by the present disclosure can realize the pipeline connection between the first device and the second device, which can avoid the entry of stray bacteria leading to the contamination of the culture fluid, etc.

The detachable connection structure between the protective cover and the main body of the connecting member and the detachable connection structure between the two main bodies of the connecting member are realized by means of a snap plate and an inner enclosure plate, which eliminates the need to set up sets of snap connection structures, and is simple in structure and convenient in production. Moreover, the connection between the connecting member body and the protective cover and between the connecting member body and the film puller does not involve a fine structure, and it is easier to open a mold as well as to produce.

1. A sterile connection member, comprising:
a main body, a first surface of the main body is connected to an external pipeline, a fluid channel is arranged inside the main body, the fluid channel penetrates from the first surface to a second surface of the main body, the second surface comprises at least one first hook and/or at least one second hook, the first hook is matched with a second hook of a main body of another sterile connection member to form a buckle connection, and/or the second hook is matched with a first hook of a main body of another sterile connection member to form a buckle connection; and
an unlocking hole formed in the main body and communicating with the buckle connection for guiding an unlocking member to press the second hook and/or the first hook at the buckle connection to release the buckle connection.

2. The sterile connection member of claim 1, wherein a first side of the second surface includes the first hook, and a second side includes the second hook, the first hook cooperates with the second hook of another sterile connection member's main body to achieve the buckle connection, and the second hook cooperates with the first hook of another sterile connection member's main body to achieve the buckle connection.

3. The sterile connection member of claim 2, wherein the first hook is an inner enclosure plate, and the second hook is a snap plate, the inner enclosure plate is provided with an inwardly extending snap strip at its end, the snap plate is provided with an outwardly extending snap hook at its end, the snap strip on the inner enclosure plate is connected to the snap hook on the snap plate of the another sterile connection member for a first buckle connection, and the snap hook on the snap plate is connected to the snap strip on the inner enclosure plate of the another sterile connection member for a second buckle connection.

4. The sterile connection member of claim 3, wherein the second side of the second surface of the main body further comprises an outer enclosure plate, the outer enclosure plate is provided on the outer side of the snap plate to limit an outward movement of the inner enclosure plate of the another sterile connection member in the buckle connection state.

5. The sterile connection member of claim 1, further comprising the unlocking member, the unlocking member laterally squeezes the second hook and/or the first hook at the buckle connection to cause it to undergo a restorable deformation in order to release the buckle connection.

6. The sterile connection member of claim 5, wherein the unlocking member is provided with a barbed side edge at the end of the unlocking member, the width of the end of the side edge is greater than the width of the unlocking hole.

7. The sterile connection member of claim 1, further comprising a film, the film is provided on the second surface of the main body to cover an opening of the fluid channel on the second surface of said body, an end of the film extends to the exterior of the main body for pulling and tearing away.

8. The sterile connection member of claim 7, wherein the end of the film extending to the exterior of the main body is provided with a film puller by which the film is pulled away from the second surface of the main body, the film puller is detachably connected to the main body.

9. The sterile connection member of claim 8, wherein the film puller is provided with at least one bump and/or at least one recess on the outer side of the film puller, wherein the at least one bump is snapped in correspondence with at least one recess on the outer side of the film puller of the another sterile connection member, and/or the at least one of recess is snapped in correspondence with at least one bump on the outer side of the film puller of the another sterile connection member.

10. The sterile connection member of claim 8, wherein the film puller is provided with at least one bump on the inner side, the main body is provided with at least one recess on the outer wall, and the at least one bump on the inner side of the film puller cooperates with the at least one recess on the outer wall of the main body to realise the snap-fit of the film puller with the main body.

11. The sterile connection member of claim 1, further comprising a protective cover, the protective cover is detachably connected to the second surface of the main body.

12. The sterile connection member of claim 11, wherein a first side of the inner side of the protective cover is provided with a splint, a gap exists between the splint and the edge of the protective cover, and the first hook extends into the gap to achieve an interference connection with the protective cover, and/or a second side of the inner side of the protective cover is provided with a holding frame, and the second hook of the main body extends into the holding frame to achieve an interference connection with the holding frame.

13. The sterile connection member of claim 1, wherein the opening of the fluid channel on the second surface of the main body is provided with a sealing member, the end surface of the sealing member is higher than the end surface of the second surface of the main body.

14. A sterile connection apparatus, comprising:
a sterile connection member, comprising:
a main body, a first surface of the main body is connected to an external pipeline, a fluid channel is arranged inside the main body, the fluid channel penetrates from the first surface to a second surface of the main body, the second surface comprises at least one first hook and/or at least one second hook, the first hook is matched with a second hook of a main body of another sterile connection member to form a buckle connection, and/or the second hook is matched with a first hook of a main body of another sterile connection member to form a buckle connection; and
an unlocking hole formed in the main body and communicating with the buckle connection for guiding an unlocking member to press the second hook and/or the first hook at the buckle connection to release the buckle connection,
wherein the first hook of the main body of the sterile connection member cooperates with the second hook of the main body of another sterile connection member to achieve a buckle connection, and/or the second hook of the sterile connection member cooperates with the first hook of the main body of another sterile connection member to achieve a buckle connection,
the unlocking hole of the sterile connection member is provided in the main body and connected to the buckle connection, for guiding the unlocking member to squeeze the second hook and/or the first hook at the buckle connection to release the buckle connection.

15. The sterile connection apparatus of claim 14, wherein a first side of the second surface includes the first hook, and a second side includes the second hook, the first hook cooperates with the second hook of another sterile connection member's main body to achieve the buckle connection, and the second hook cooperates with the first hook of another sterile connection member's main body to achieve the buckle connection.

16. The sterile connection apparatus of claim 15, wherein the first hook is an inner enclosure plate, and the second hook is a snap plate, the inner enclosure plate is provided with an inwardly extending snap strip at its end, the snap plate is provided with an outwardly extending snap hook at its end, the snap strip on the inner enclosure plate is connected to the snap hook on the snap plate of the another sterile connection member for a first buckle connection, and the snap hook on the snap plate is connected to the snap strip on the inner enclosure plate of the another sterile connection member for a second buckle connection.

17. The sterile connection apparatus of claim 16, wherein the second side of the second surface of the main body further comprises an outer enclosure plate, the outer enclosure plate is provided on the outer side of the snap plate to limit an outward movement of the inner enclosure plate of the another sterile connection member in the buckle connection state.

18. The sterile connection apparatus of claim 14, further comprising the unlocking member, the unlocking member laterally squeezes the second hook and/or the first hook at the buckle connection to cause it to undergo a restorable deformation in order to release the buckle connection.

19. The sterile connection apparatus of claim 18, wherein the unlocking member is provided with a barbed side edge at the end of the unlocking member, the width of the end of the side edge is greater than the width of the unlocking hole.

20. The sterile connection apparatus of claim 14, further comprising a film, the film is provided on the second surface of the main body to cover an opening of the fluid channel on the second surface of said body, an end of the film extends to the exterior of the main body for pulling and tearing away.
